(19) United States
(12) Patent Application Publication
(10) Pub. No.: US 2009/0240134 A1
(43) Pub. Date: Sep. 24, 2009

(54) Method of processing thoracic reflected radio interrogation signals

(76) Inventors: Andrew Pal, Las Vegas, NV (US); Ann K. McCaughan, Les Vegas, NV (US)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(21) Appl. No.: 12/383,362

(22) Filed: Mar. 23, 2009

Related U.S. Application Data
(63) Continuation-in-part of application No. PCT/US07/20487, filed on Sep. 21, 2007.
(60) Provisional application No. 60/846,404, filed on Sep. 21, 2006.
(60) Provisional application No. 60/973,988, filed on Sep. 20, 2007.

(51) Int. Cl. A61B 5/05

(52) U.S. Cl. 600/407

(57) ABSTRACT

A method of evaluating or monitoring the medical state of a subject comprising the steps of: providing a radio frequency interrogation interference signal from a subject, the radio frequency interrogation interference signal being low frequency components of reflections of a radio interrogation signal transmitted into the thorax of the subject; and determining at least one cardiac or respiratory characteristic of the subject from radio frequency interrogation interference signal. The characteristic can be compared to predetermined values to assess current state or monitored for changes over time to assess change in condition of the subject.

### CROSS REFERENCE TO RELATED APPLICATIONS

The present application is a continuation-in-part of PCT/US2007/020487 which was filed Sep. 21, 2007 entitled “Method for Processing Thoracic Reflected Radio Interrogation Signals” and claims priority from U.S. Patent Application No. 60/846,404 entitled “Method of Processing Thoracic Reflected Radio Interrogation Signals”, filed Sep. 21, 2006 and U.S. Patent Application No. 60/973,988 entitled “Method of Processing Thoracic Reflected Radio Interrogation Signals”, filed Sep. 20, 2007, all of which are incorporated by reference herein in their entireties

### STATEMENT REGARDING FEDERALLY SPONSORED RESEARCH OR DEVELOPMENT

The U.S. Government has a paid-up license in this invention and the right in limited circumstances to require the patent owner to license others on reasonable terms to the extent and under the provisions as provided for by Contract No. DAH001-05-S-0144 awarded by the U.S. Air Force Special Operations Command (AFSOC).

### BACKGROUND OF THE INVENTION

In biomedical engineering, thoracic bioimpedance is a measure of changes in the electrical conductivity of the thorax and heart. The measurement is based on pulsatile blood volume changes in the heart and aortic root. First described in 1959 by Kubicek, this approach to hemodynamic measurement has been refined and used in practice since the early 1990's. Thoracic electrical bioimpedance (TEB) noninvasively measures rate, power and volume associated with the cardiac cycle. Validation studies have correlated the results of non-invasive thoracic impedance measurement with the invasive Swan Ganz Thermodilution measurement as well as the invasive Fick method of measuring cardiac output.

Existing, invasive methods of cardiac output and hemodynamic monitoring (Swan Ganz or Fick procedures) are not available or practical for use outside medical facilities. Even existing non-invasive thoracic impedance devices are impractical for field use, due to size and power requirement and the need to connect multiple (e.g. seven) electrodes to the patient's chest.

### BRIEF SUMMARY OF THE INVENTION

In one aspect, the invention is a method of evaluating or monitoring the medical state of a subject comprising the steps of: providing a radio frequency interrogation interference signal from a subject, the radio frequency interrogation interference signal being low frequency components of reflections of a radio interrogation signal transmitted into the thorax of the subject; and determining at least one cardiac or respiratory characteristic of the subject from radio frequency interrogation interference signal.

In another aspect, the characteristic can be compared to predetermined values to assess state or monitored for changes over time to assess change in condition of the subject.

In yet another aspect, the invention is a method of processing cardiopulmonary radio data obtained by transmitting a radio interrogation signal into a subject torso and capturing reflections of the radio interrogation signal from various tissue of the torso. The method comprises the steps of: extracting time varying components from the captured reflections of the radio interrogation signal as a radio interrogation impedance signal; and extracting cyclical respiratory component from the radio interrogation impedance signal.

### BRIEF DESCRIPTION OF THE DRAWINGS

The foregoing summary, as well as the following detailed description of the invention, will be better understood when read in conjunction with the embedded figures. For the purpose of illustrating the invention, there are shown in the figures embodiments which are presently preferred. It should be understood, however, that the invention is not limited to the precise arrangements and instrumentalities shown.

FIG. 1 depicts diagrammatically, radio signal reflection surfaces within the torso of a human subject;

FIG. 2 depicts an exemplary trace of a typical Radio Frequency Impedance Interrogation signal with cardiac points of maximal impulse (“C”) indicated;

FIG. 3 is a simplified trace of the RFII signal of FIG. 2 collapsed in time to illustrate a respiratory component (L) of the RFII signal;

FIG. 4 is a reproduction of the trace of FIG. 2 illustrating a respiratory component baseline stretched over several consecutive heartbeats;

FIG. 5 is a reproduction of the trace of FIG. 3 showing the full cyclical nature of the respiratory component of the RFII signal;

FIG. 6 is another detailed portion of the trace of FIG. 2 to illustrate the slope that exist between the immediately adjoining extrema;

FIG. 7 is an exemplary trace indicating the impact of exaggerated breathing on the RFII signal;

FIG. 8 depicts traces illustrating one respiratory contribution that is reflected a cardiac cycle of the RFII signal for three different subject;

FIG. 9 depicts traces illustrating another respiratory contribution that is reflected in a cardiac cycle of the RFII signal for the same three different subjects of FIG. 8;

FIG. 10 illustrates a RFII signal trace covering two heartbeat with respiratory component removed and the change in reflectivity (amplitude) over time;

FIG. 11 illustrates simultaneously generated RFII cardiac component signal and conventional conductive impedance generated dZ/dt signal superimposed upon one another.

FIG. 12 depicts the RFII and dZ/dt traces of FIG. 11 separated from one-another, and the amplitude of the RFII cardiac component signal inverted;

FIG. 13 depicts the traces of FIG. 12 combined in time synchronization and various cardiac landmarks from the ECG signal;

FIG. 14 depicts a similar pair of traces for a subject with a left bundle branch block;

FIG. 14a depicts in greater detail the intersection of the two traces at a T-wave in FIG. 14;

FIG. 15 is an enlarged version of a single RFII wave with a single corresponding ECG cardiac event from FIG. 13;

FIG. 16 is an enlarged version of a single RFII wave with a single corresponding ECG cardiac event from FIG. 14 with a left bundle branch block;

FIG. 17 illustrates an identifies other components of the RFII signal that may be used for quantification or evaluation of respiration of a subject;

FIG. 18 is an RFII trace that identifies yet additional respiration subcomponents of the reflected RFII cardiac component signal;

FIG. 19 is a flow chart illustrating a method of heart rate and respiration rate calculation from the RFII signal; and

FIG. 20 is a flow chart of the calculation of dR/dt from the reflected RFII signal.

### DETAILED DESCRIPTION OF THE INVENTION

Hemodynamic and bioimpedence states occur every heartbeat, or as a subcomponent of a beat. Each action within the heart causes a physical mechanical and electrical change. Although the repetition of these actions can be viewed as a general average over a period of time, each heartbeat is a unique event, and thus contains a unique set of values and characteristics that provide unique information.

Concurrently, external forces exert themselves onto these values and components of a heartbeat causing variations. These variations are generally very small and do not inject major deflections in the data ranges. External forces may include, but are not limited to the subject's physical position such as standing or sitting. Breathing rate changes can provide a variance as well. Physical exertion, environment, and overall physical state also affect these parameters.

Therefore when sudden changes occur, or deviations within a trend occur, an evaluation can be made as to the condition of the individual. When compared to the initial values or individual baseline, a determination may be made as to general health. Additionally, by predicting a future trend from the collected trend data, a patient can be evaluated in terms of future outcome. This includes improvement, stability or decline in the health of the monitored individual. Therefore therapeutic intervention may be guided in order to prevent decompensation and improve outcomes.

Conventional non-invasive cardiac impedance measures electrical conductivity within an individual between at least two sensing electrodes which are attached to a subject between two additional, interrogation signal injecting electrodes. It has been discovered that the reflected radio waves contain much if not all of the same hemodynamic data as is contained in conventional non-invasive thoracic bioimpedance signals. It has been further discovered that radio signals can be safely transmitted to penetrate a subject's torso and be reflected to varying degrees from various internal thoracic organs with enough strength and frequency variability to provide cardiological data like that obtained in conventional contact impedance signals as well as respiratory data not readily available in such signals. The present invention will be referred to generically by the acronym RFII for Radio Frequency Impedance Interrogation.

A radio interrogation signal is transmitted into the torso of a subject through an antenna positioned proximal to the subject. However, unlike conventional non-invasive thoracic bioimpedance measurements, the antenna does not have to be in direct contact with the subject, just sufficiently near to the subject opposite the subject's heart. As the transmitted radio interrogation signal passes through with the subject, various factors affect the interaction of the waves with the subject and provide investigational information.

Consider a single source of power in the form of an unmodulated radio signal having a predetermined fixed frequency which is transmitted from an antenna into the torso of a unmodulated subject positioned with the antenna and which reflects off of all thoracic organs and substances encountered. The major radio wave reflecting substances encountered by the radio interrogation signal are depicted diagrammatically in FIG. 1. Although this is a simplified image with several components missing, it does reflect the major influences on the transmitted radio interrogation signal. The radio interrogation signal landmarks are: D1 (Derma); M1 (Muscle); S1 (Skeletal); L (Lung); CM (Myocardium); CF (Cardiovascular Fluid); S2 (Skeletal); M2 (Muscle); and D2 (Derma).

When radio interrogation signal is transmitted into the subjects' torso, the first deflection/reflection occurs on the skin (derma), followed by muscle followed by bone (the ribcage). As these three physical components are static in size and composition, the returned reflections of the radio interrogation signal from these components is a steady value.

Once the radio interrogation signal passes the ribcage, a portion of it reflects from the lungs. As the lungs expand and contract, the composition of the organ changes. When the lungs are exhausted of air, the bulk of the material within the volume of the lungs is tissue. The tissue contains salt and water. Salt water, including blood, is one of the most reflective materials to the radio interrogation signal. Salt water/blood causes the reflected signal to increase.

The radio interrogation signal portion which passes into and beyond the lungs comes into contact with the heart where a portion of the radio interrogation signal is reflected back. This portion provides a constant value as the composition of the muscle itself remains relatively constant. However, the radio interrogation signal which enters the heart is modified by a number of factors resulting in a varied signal return which can be monitored and evaluated. When the heart proceeds through its cardio-dynamic process, the shape and displacement of fluid levels changes. The shape of the heart changes as well. All of these physiological events modify the signal which is reflected back out of the body. Not only does the volume of fluid affect the signal reflection, its shape as it is defined by the container (the heart and blood vessels) provide a unique, consistent pattern of the reflected signal. The radio interrogation signal which reflect the fluid volume, have the opportunity to be reflected from the back or opposite surface on the fluidic shape. These reflections are smaller components. However, they affect the characteristics of the signal uniformly on a beat to beat basis due to the continuity of the fluidic shape as it appears within the heart.

Beyond the heart, the balance of the radio interrogation signal will again come into contact with bone such as the spinal column followed by muscle and skin. During this process, portions of the radio interrogation signal will be reflected back in a consistent manner as the composition and shape of these materials remains static during the process.

The lungs, myocardium and cardiovascular fluid experience the greatest amount of change cyclically. The lungs develop an atmosphere pocket with a specific shape based on internal topology which is relatively unique on a person by person basis. These atmosphere pockets create surfaces which reflect radio interrogation signals back and forth and provide alternating periods where higher reflectivity and lower reflectivity occur. The heart develops a fluid pocket (cardiovascular fluid) which moves and changes shape as it travels through the organ. The fluid (blood) is highly reflective of radio waves.

Not only is the radio interrogation signal affected by the reflectivity of the anatomical structures and bodily substances that it encounters, but it is also affected by the position of the heart and how it is positioned in proximity to the other anatomical structures. This is evidenced by signals obtained from individuals with barrel chests and large pectoral masses as opposed to thinner, more lean individuals. The reflected signal is not dampened or enhanced by body composition, but has a different slope. The reflections of the radio interrogation signal from each subject have properties that relate to anatomical positioning, anatomical shape and size, mechanical action, and the electrical conductivity/bioimpedance properties of each of the thoracic structures and substances encountered.

When analyzing the reflections of the radio interrogation signal, the Doppler components contain the cardiopulmonary information of interest. Hereinafter the Doppler components of the captured reflected radio interrogation signal will be referred to as the reflected Radio Frequency Impedance Interrogation signal or simply “RFII” signal. The Doppler components of interest are in the range of about 100 Hz and less. The RFII signal that is being processed represents the amplitude of the captured reflected radio waves in that Doppler bandwidth around the predetermined fixed frequency of the original radio interrogation signal.

At any given time T, the morphology of the subject is a static snapshot of it's behavior and characteristics. As it is a static snapshot at a given moment, bodily fluids such as blood should not be viewed or interpreted as a liquid, but rather as a three-dimensional solid existing in a static state at that given moment. Even though the radio wave is in motion and decaying in strength as it expends energy, at the given time T it is best to interpret the fluid as a singular solid object with reflective surfaces and angles of incidence. In order to determine velocity and/or direction of the wave, or energy motion, multiple time slices of information must be evaluated.

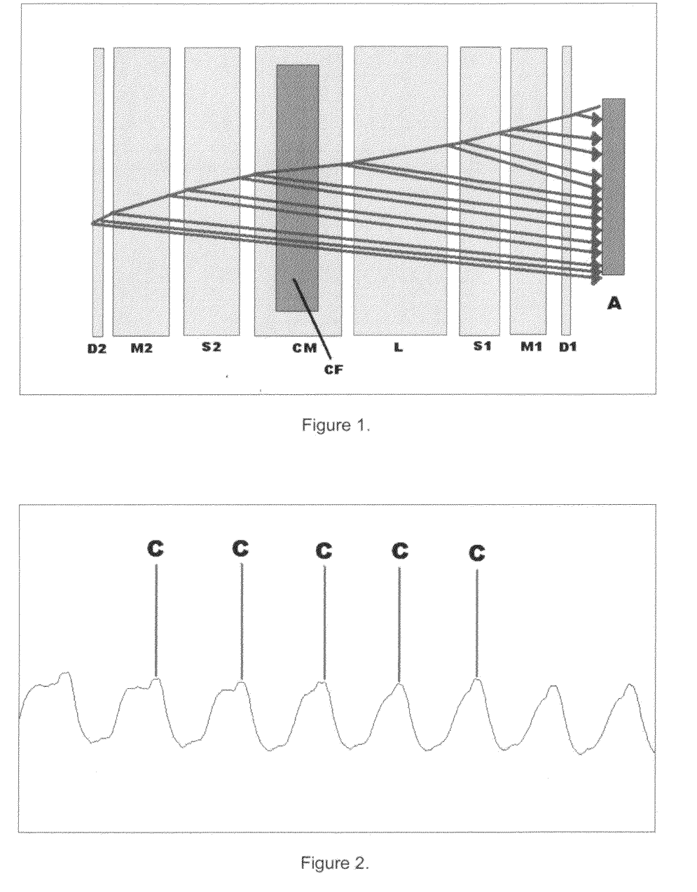

FIG. 2 depicts an example of a trace of an RFII signal from a subject. Again, this signal is the amplitude of the Doppler components of the captured reflections of the radio interrogation signal. This RFII signal can be obtained from the raw reflected returns in various ways but quadrature demodulation and band filtering (between about one to one hundred hertz) are preferred. The simplest and most obvious components within the traces of the RFII signal depicted in FIG. 2 are the cardiac landmarks (“C”). The heart operates with a cyclical fluidic change. As the fluid is highly reflective for radio signals like the original radio interrogation signals, this interaction becomes quite pronounced. As the heart fills with fluid (blood), the signal response increases returning a topological representation in a one dimensional data array. Easily identified, the cycle of contractions (heartbeats) are determined by counting these variations (C events) within a specific time period.

Another cyclical system which occurs concurrently within the RFII signal is respiration. Although it is not as obviously apparent as the heartbeats, it is present within the RFII signal. Ideally to evaluate the signal from the heart in greater detail, the respiratory signal component should be removed.

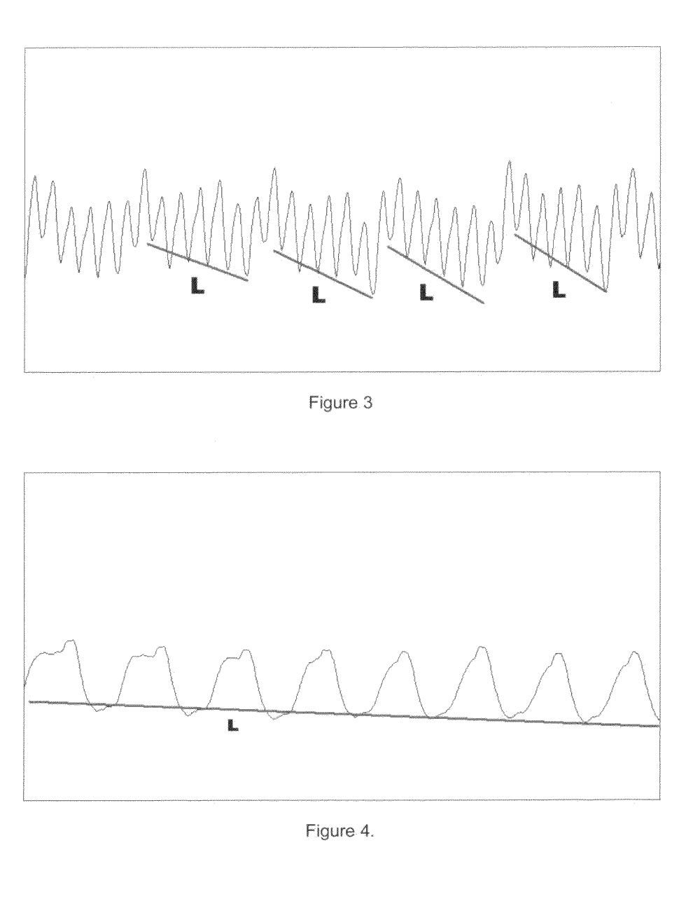

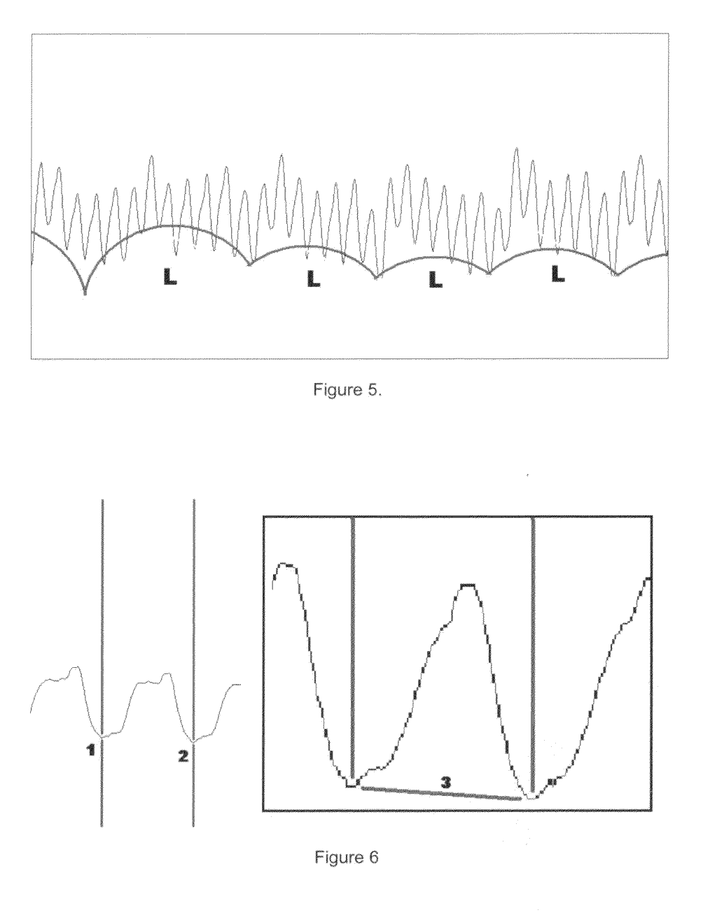

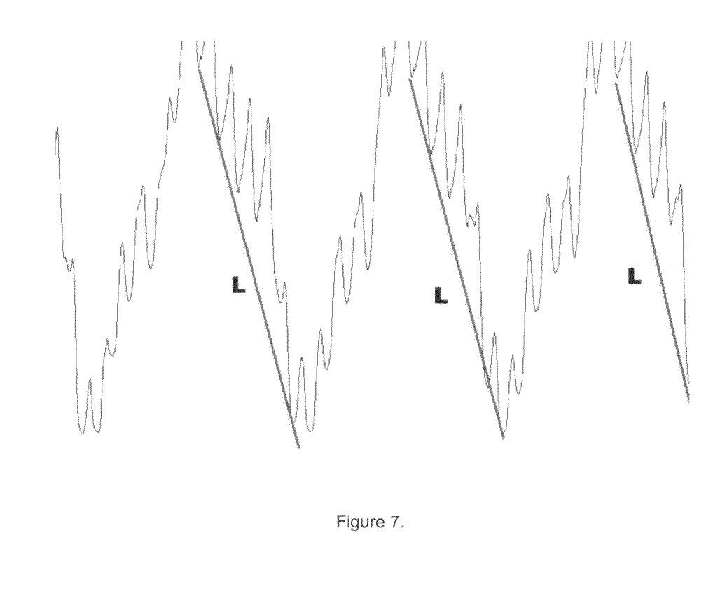

In FIG. 3, about twenty seconds of the RFII signal is depicted to illustrate a normal respiratory cycle. The diagonal lines “L” show the rhythmic breathing pattern superimposed on the cardiac cycle by respiration. The internal topology of the lungs as well as their constitution affect their reflective abilities of the RFII signal. When empty, the lungs are more reflective as fewer surfaces are exposed to provide incidental angles of deflection as well as incidental angles of refraction for the RFII signal. When this is the case, more RFII signal is able to be reflected back towards the source. When the lungs filled with air, the bronchial tree and alveoli expand creating surfaces which provide angles in incidence for reflection as well as refraction of the RFII signal. During this phase, the strength of the reflected signal reduced. If the RFII signal from FIG. 2 is closely reexamined, the respiratory subcomponent signal (L) of breathing becomes apparent as indicated in FIG. 4. The respiratory cycle creates a reoccurring baseline rise and fall as illustrated in FIG. 5, where the RFII of the previous figures are time compressed. The respiratory cycle can be quantified by counting the bottoms and/or tops of the underlying cycle as it appears within the RFII signal to provide a respiratory rate L in respirations per minute. This component can also be removed for isolation of the signal from the heart.

In FIG. 6, a slope “3” exists between points 1 and 2 within consecutive extrema of the RFII signal. Points 1 and 2 provide convenient anchor points for the removal of the base breathing component. As the reflected RFII signal is not affected in a linear manner by respiration, it is important to remove this component with a segmented approach. Utilizing the heart beat as a point of reference, a slope 3 between points 1 and 2 is determined for the increase or decrease of the baseline. This slope is systematically removed from all points between 1 and 2. This process is then repeated for the following pair of heartbeats, between 2 and the following point of reference (not depicted). When inhaling and exhaling are exaggerated, the baseline deflections and subsequent underlying amplitude of the breathing wave becomes more pronounced and exaggerated FIG. 7 depicts a trace of an exemplary RFII signal where very deep breathes are taken by the subject generating a steeper slope. Alternately, the figure can reflect an exhalation effort to exhaust as much air as possible to from the lungs. The breathing cycle becomes very pronounced and creates a signal with a higher range of deviation than the heart beat.

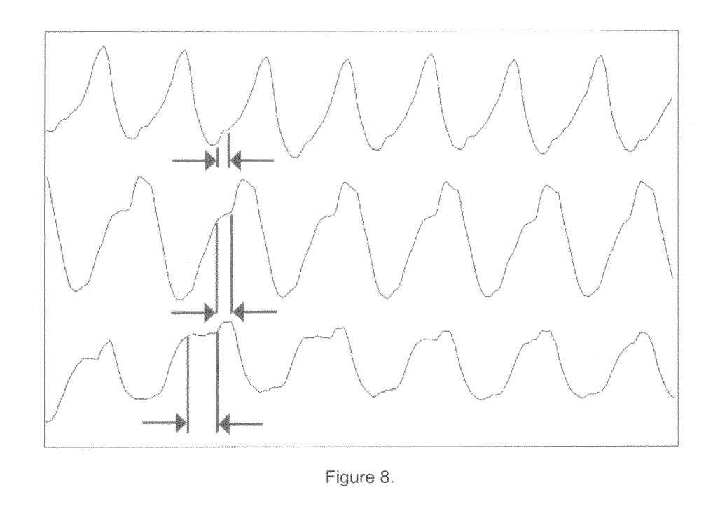

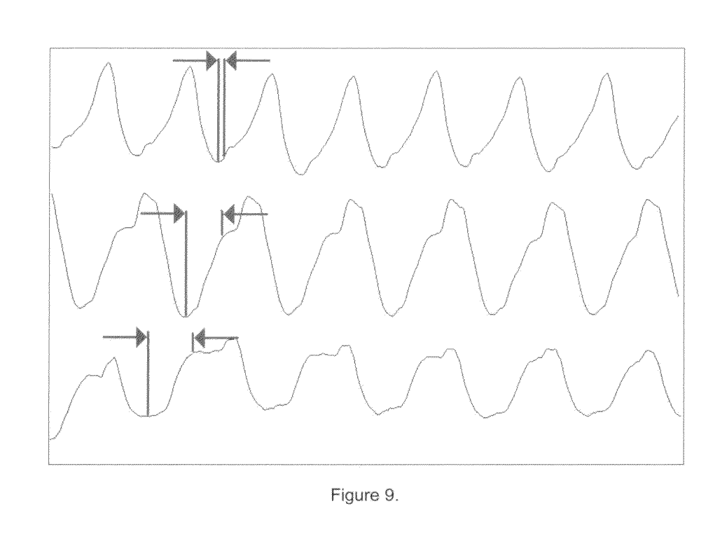

Another pulmonary feature exists within the reflected RFII signal. A residual RFII echo occurs within the lung as well as a “drum mechanic”. As the lungs fill with air, they provide multiple surfaces which amplify the echoing characteristics of the RFII signal in addition to the reflected signal response decline. A pronounced characteristic becomes apparent within the signal as can be seen in FIG. 8 depicting RFII signals from different subjects. The top line within this set of three RFII traces is from one exhaled duration. The small feature indicated at the leading edge of the heartbeat corresponds to residual air within the lungs, which may be interpreted as the Expiratory reserve volume under normal breathing or as the Residual volume after maximal exhalation. Signal deviators such as liquid in the lungs would most likely create a significant signal event for measurement. Additional respiratory and/or information exists within the feature which can be utilized to ascertain the condition of the lungs as can be seen in FIG. 9. Three separate portions of a trace are assembled above one another in FIG. 9 and depict the cyclical modification of the signal over time for different subjects as the lungs expand and contract.

As the respiratory cycle related components are removed from the reflected RFII signal, the residual signal which exists constitutes clothing, skin, muscle, bone, static lung material, the heart muscle and fluid (blood). The primary dynamic component within this set of items is the fluid blood as it changes cyclically. As the fluid (blood) influences the magnitude of the reflected signal, volumes can be determined based on a response. Additional information regarding the health of the heart can be determined by various aspects of the residual signal which shall also be referred to as the RFII cardiac component.

When the RFII signal and particularly the RFII cardiac component is compared with a simultaneously generated thoractic electrical bioimpedance signal, a correlation can be seen to exist between the conventional conductive impedance signal dZ/dt and RFII cardiac component signal. Both are significantly influenced in similar ways by fluidic volume and its changes.

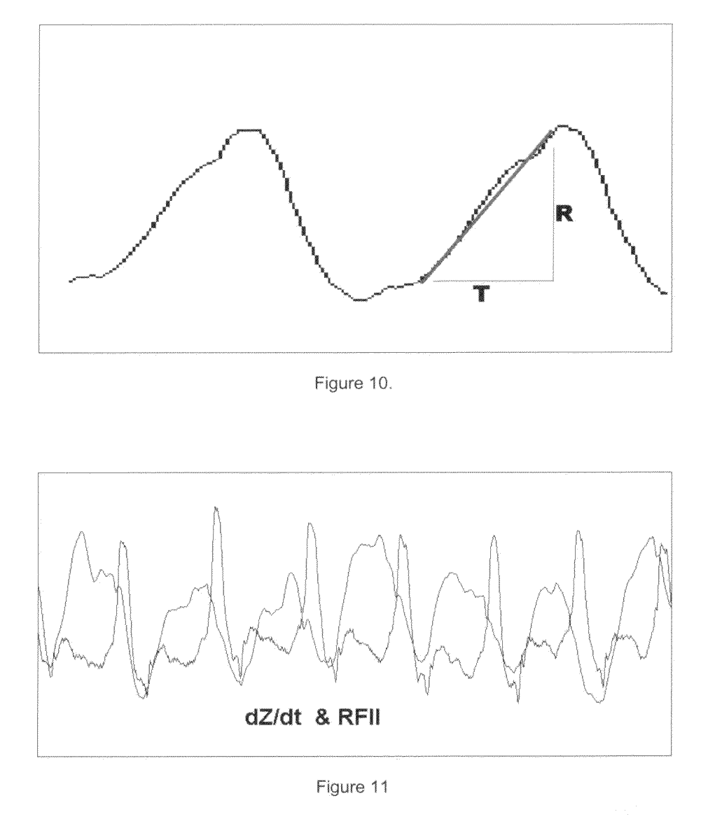

In FIG. 10, a single heartbeat from the (inverted) RFII cardiac signal is evaluated for amplitude and time changes. This has been found to provide a value for this cardiac characteristic comparable to the thoracic impedance characteristic value dZ/dt. This cardiac characteristic determined from the RFII signal can be viewed as the reflectivity change in relation to the time change (dR/dt).

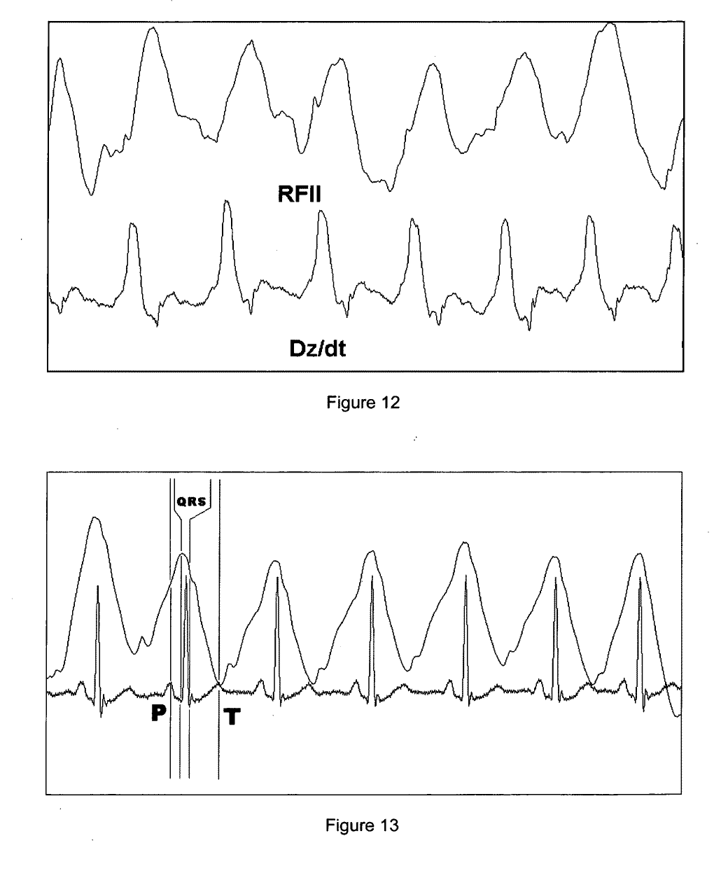

FIG. 11 represents an exemplary screen capture of simultaneously generated RFII cardiac component and conventional conductive impedance generated dZ/dt signals superimposed upon one another. The timing and basic shape of the two signals can be compared. In FIG. 12, the reflected RFII cardiac component and the dZ/dt signals of FIG. 11 have been separated and the amplitude of the reflected RFII cardiac signal is inverted with a non inverted dZ/dt signal underneath. FIG. 13 shows an inverted, reflected cardiac RFII cardiac signal (top) with a concurrent ECG signal (bottom).

The characteristics of the leading and trailing slopes of both signals in FIGS. 11 and 12 present similar characteristics in reference to amplitude versus time. It has been found that the similarity between dR/dt shown in FIG. 10 and dZ/dt can be used to determine an at least relative value of Stroke Volume (“SV”) and Cardiac Output (“CO”) for a subject directly from the RFII signal. The value dR/dt can be determined in various ways. A simple expedient is to measure signal amplitude between minima to maxima points in one cardiac cycle of the RFII signal, determine the time duration of the period and use that number as a dZ/dt equivalent in the conventional Stroke Volume and Cardiac Output equations. While appropriate scaling would be necessary for an absolute determination of each of these two characteristics, it will be appreciated that for trend monitoring or gross characteristic determination, scaling would not be important. A nominal SV and/or CO value or a nominal range of such values can be determined by measurement of various subjects using dR/dt values and the value of an individual's SV and/or CO value compared to the nominal value(s) for a determination of subject condition. A more accurate determination can be made by calculating an average dR/dt value over several sequential cardiac cycles. It is important to note that for greater accuracy, the breathing component should be removed from these portions of the RFII signal (FIG. 6). Otherwise the time portion would increase during inhalation thereby creating an unusable value for several beats per cycle skewing the resultant values.

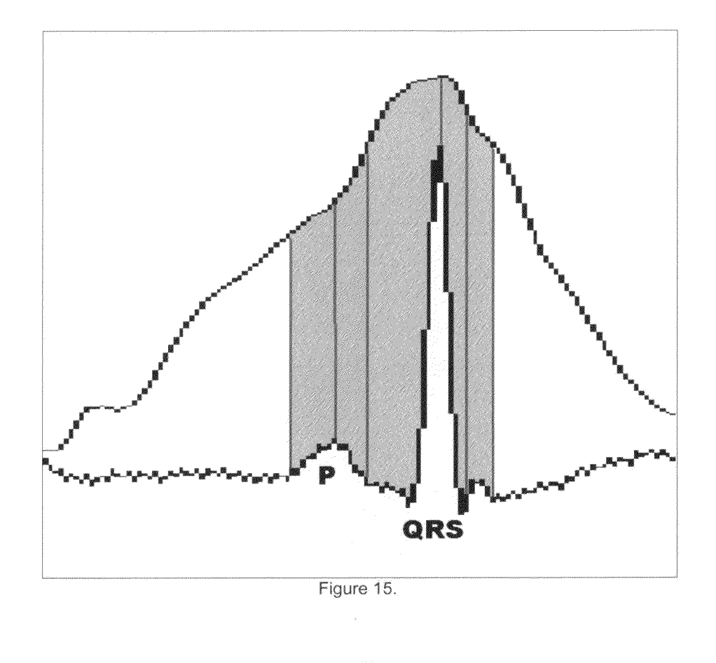

Additionally, minute features within the dZ/dt signal at certain events are amplified and present more prominent features within the inverted RFII cardiac component signal. The timing of known events such as cardiac waves in the ECG can be utilized to further identify the same events and other characteristics from the RFII cardiac component signal. Referring to FIGS. 13 and 15, the normal P wave which occurs within an ECG correlates precisely with a RFII cardiac slope deviation which is evident prior to the top extrema of the inverted RFII cardiac component signal. The QRS complex is constantly visible within the inverted RFII cardiac signal as the data segment beginning with the top of the peak extending to the visible deflection on the declining slope. When the single heartbeat capture from FIG. 13 is magnified in FIG. 15, the correlations between the ECG event times and the RFII event times become more apparent. The T wave corresponds with the bottom on the event trough within the inverted RFII cardiac signal. By utilizing a matching ECG, significant events are discernable within the RFII cardiac signal. As a result, these cardiac event or landmarks, which constitute subcomponents within each cardiac cycle, can be identified and used to determine those cardiac characteristics normally requiring ECG data. For example, characteristics such as Ventricular Ejection Time (VET) can be determined from the RFII signal.

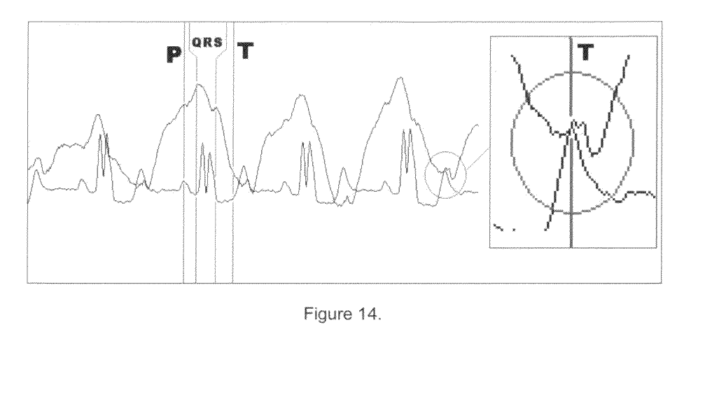

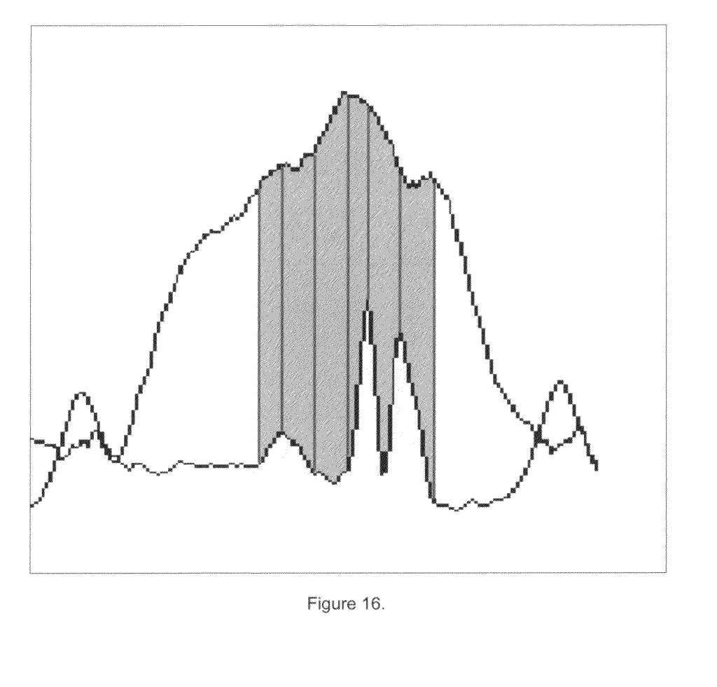

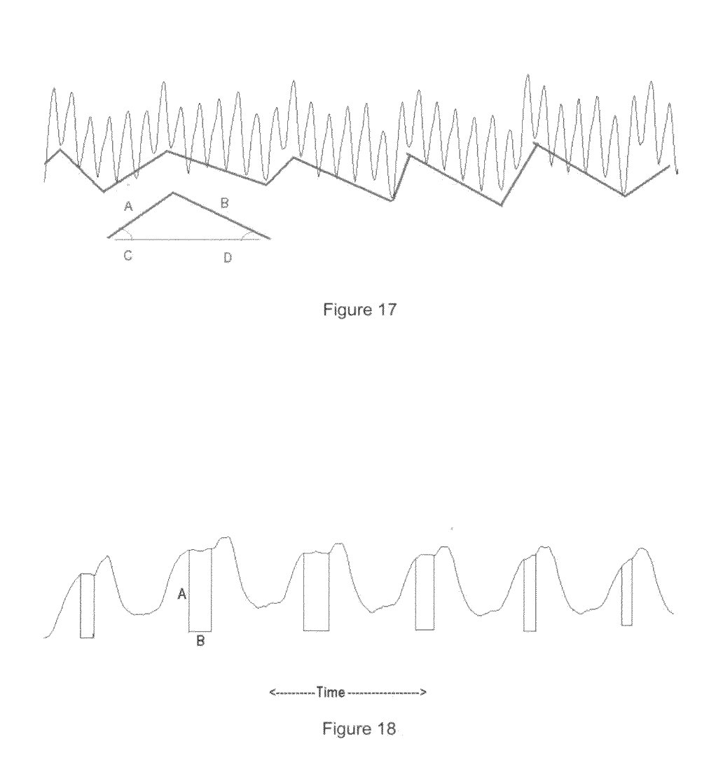

In FIG. 14 concurrent ECG (bottom) and inverted, RFII cardiac component (top) signals show the relationship between the signals when the conditions in the heart are not optimal, for example, with a left bundle branch block. When the single heartbeat from FIG. 14 is magnified in FIG. 16, the timing events between the ECG and RFII become very apparent. The corresponding slope deviation at the point of the P wave creates a discernable peak within the RFII signal. Although the P wave on the ECG is not dramatically out of proportion to a normal P wave, the mechanics of the heart and fluid present a noticeable deviation at that time period. Instead of a slope deviation as can be seen in FIG. 13, the deviation begins with a signal spike followed by a signal drop. Additionally the QRS complex signal signature in the RFII signal deviates from a normal RFII cardiac signal. The trailing slope change is moved further away from the peak on the time scale and is significantly more pronounced. When no breathing is taking place and the lungs are depleted of air, the RFII signal presents a dip in the signal. This event occurs at the corresponding time as the trailing deviation in the QRS complex. During the up-slope of the T wave, the bottom of the RFII signal changes from a uniform trough which appears U shaped to an unbalanced trough. The declining curve is sloped in a shallower manner. These changes in the RFII cardiac component can be used to identify the occurrence of these various cardiac events and conditions

FIG. 17 depicts RFII signal elements that are components of a detailed analysis of the heart. In the above signal trace, the respiration wave has components which can be utilized for quantification or evaluation of the subject's pulmonary/respiratory response. These include the following. The length A of the slope on the rising edge of the inverted RFII signal wave indicates depth of breath. Length B, the length of the declining slope of the falling edge of the RFII signal wave, is the exhalation portion of the respiratory cycle. The angle AC is the slope of A with respect to C and represents the depth of the inhalation. The deeper the inhalation, the greater the lung expansion and the steeper the slope. Slope times duration of the inhalation gives an indication of intake volume. Any of the three (respiratory rate, depth of breath and indication of intake volume) can also be monitored for change.

Referring as well to FIG. 18, additional respiration subcomponents include but are not limited to the extraction of the pulmonary generated section within each heartbeat, which is also directly related to the heart and it's mechanics and responses. These include, for example, the identification of the point in the heartbeat component where the slope of the cardiac wave levels out, which is due to pulmonary activity; the identification and evaluation of the end of the event where the slope again rises; the duration of the event as denoted as item (B) and seen in FIG. 18 to vary cyclically within the heartbeat waves as a measure of respiratory rate; calculation of (A) multiplied by (B) in order to determine volume of the identified signal event as an indication of magnitude or volume of lung capacity, (B) divided by (A) and/or (A) divided by (B) in order to determine a ratio within the identified signal event; and identification within the (B) length of the cardiac component signal of specific events which correlate to the electrically identifiable physical events within the heart as indicated in FIGS. 13 and 14.

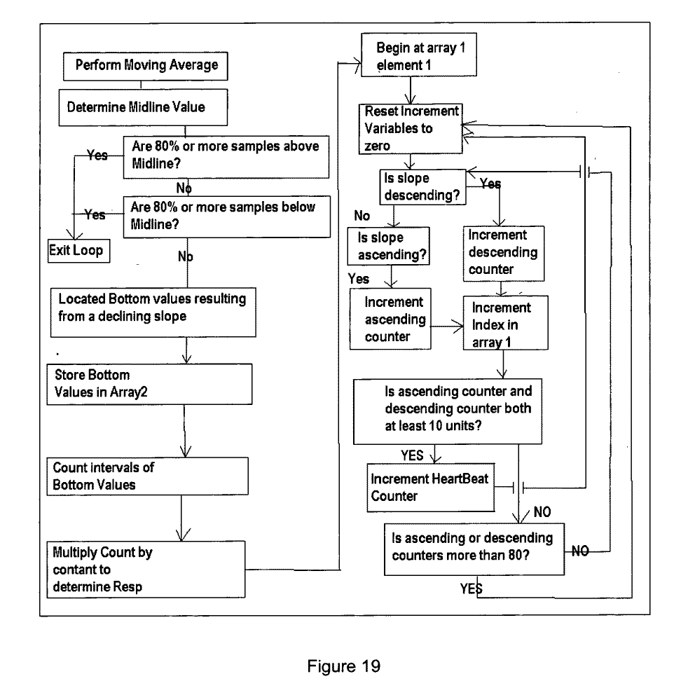

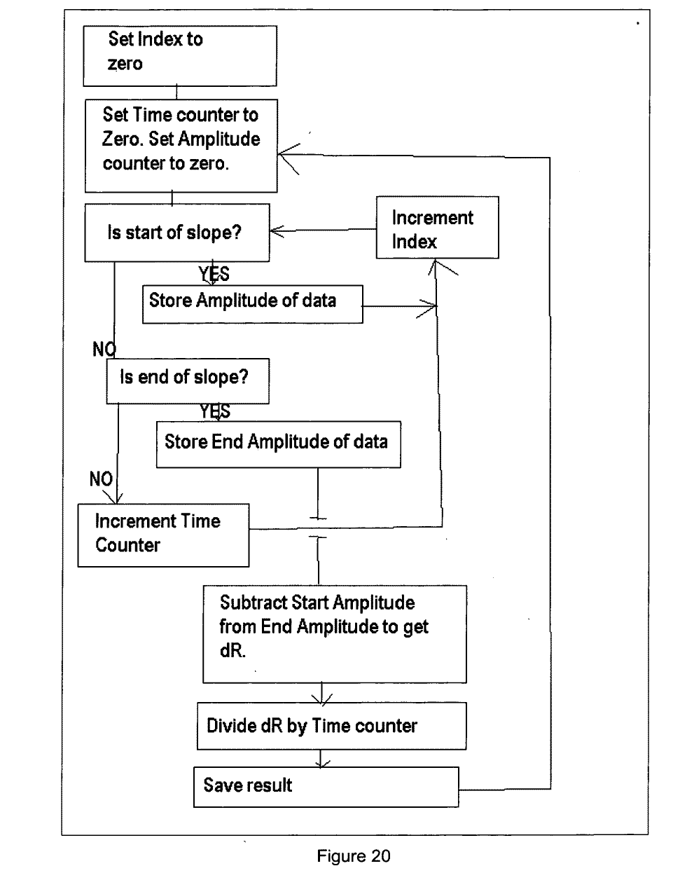

The FIG. 19 is a flow chart that illustrates heart rate and respiration calculation from the RFII signal. The flow chart of FIG. 20 depicts how dR/dt can be calculated from the cardiac component of the reflected RFII signal. As can be seen, dR/dt is determined from sequential blocks, for example, twenty to thirty second lengths of the RFII cardiac component signal.

The calculated dR/dt value can be used to determine Stroke Volume (SV) and cardiac Output (CO) with the following equations:
If male:

SV=((0.418−(0.0016*HR))*134*dR/dt*(Len*Len*Len))/(ZO*ZO)

CO=(SV*HR)/1000;
If female:

SV=(0.418−(0.0017*HR))*112*dR/dt*(Len*Len*Len)/(ZO*ZO)

CO=(SV*HR)/1000;

Where the following are used:
HR—Heart Rate
Len—Thoracic length (nominally 13 inches for male, the same or less for a female)
ZO—Baseline value of reflections from the original radio interrogation signal derived from reflected returns of the radio interrogation signal, in particular, the magnitude of the DC component of the reflections from the original radio interrogation signal; (nominal value 25 ohms male or female).

Thus is provided a method of evaluating or monitoring the medical state of a subject that begins with the steps of: providing the radio frequency interrogation interference signal from a subject, the radio frequency interrogation interference signal being a low frequency component of reflections of a radio interrogation signal transmitted into the thorax of the subject; and determining at least one cardiac or respiratory characteristic of the subject from radio frequency interrogation interference signal. The characteristic can be compared to predetermined values to assess state or monitored for changes over time to assess change in condition of the subject.

Each of these cardiopulmonary characteristics, respiration rate, heart rate, Stroke Volume and/or Cardiac Output, and others can be determined in sequential time segments of the RFII signal. Any of these determined characteristics can be outputted in real time in signal form as a printout or a visual display and/or stored for historical purposes. Changes in any of these characteristics compared with the characteristics of earlier sequential time segments or comparison of determined/calculated/derived characteristics exceeding predetermined limits or rates of change in any of these characteristics exceeding rates of change limits, can be identified in addition or in the alternative and stored or outputted in signal form. Several of these characteristic can be monitored to identify an overall condition (e.g. good, serious, critical) and changes in condition (improving, stable or deteriorating) of the subject identified. The determined condition can also be outputted in signal form. In the event of deterioration or deterioration of a sufficiently significant degree, an appropriate alarm signal can be generated.

One example of a method for monitoring condition and condition changes is to quantify or determine the value of and monitor changes in overall condition by monitoring values and changes in Cardiac Output. For example, Cardiac Output can be calculated per the above equation. The ranges for CO from the above equation should be 0 (expired) to about 12 (being an unhealthy, hyperactive heartbeat). The normally healthy range is about 3.5 to 6.5.

To put this into a more easily manipulated centiary scale of 0-100, the Cardiac Output can be multiplied by 10 (i.e. a cardiac output of 2.1 becomes 21). If the result is below 21, a value of 5 can be added to it. This is a “Emergent” situation (very unhealthy). If the result is between 21 and 26, a value of 44 can be added to it. This is “Urgent” (not healthy at the moment). If the result is above 26, a value of 44 can be added to it (it will be in the range of “Urgent” to “full health”). If the result value is greater than 100, it would be capped at 100 (full health). This value can be output to the user, compared with prior values for trending and/or stored for later comparison.

If the trend is towards lower numbers (regardless of current score) and has reduced by 10% in two or more subsequent entries in the array, it can be tagged as “Urgent” and capped at 65. If above 65, it can be brought down to 65 to flag a bad trend. If the trend is towards lower numbers (regardless of current score) and has reduced by 15% or more for two or more subsequent entries, it can be tagged as “emergent” and capped at 35. Again, if above 35 it can be brought down to 35 to flag serious deterioration. This is as an example only. This shows how cardio-pulmonary/respiratory data from the RFII signal can be used to broadly classify subjects such as patients or casualties in a way that provides an indication of current condition and/or the trend of that condition.

Instead of cardiac output, stroke volume or respiratory rate or heart rate or a cardiac wave event or other cardiac or respiratory characteristic or some combination of characteristics can be quantified and compared with prior values of the subject or with predetermined nominal values applicable widely to subjects.

Since the collection of RFII data can be done relatively quickly and easily, it is ideally suited for use in emergency situations and/or in situations where one or a limited number of care givers need to monitor the condition of many seriously ill or injured individuals. The condition and/or trend can be can be converted into a signal and provided to the care giver(s) by display on the RFII data collection apparatus, for example by the use of one or more light sources like color coded LED's and/or can be displayed continually or operated in different duty cycles of duration and/or intensity to indicate state and/or change of state. Sound signaling can be used as well, for example as an emergency alarm. Alternatively, values can be transmitted to a receiver on the care giver or someone or thing directing the care giver to assess and/or monitor patient condition. In the latter case, the transmitted values would have be sent with some type of identifier to identify the subject source.

The invention further includes extracting a cardiac function signal or pulmonary function signal from the radio frequency interrogation interference signal and processing the extracted function signal to derive subcomponents of cardiac cycles or respiratory cycles to determine intra cycle events produced by physiological changes during each such cycle.

Furthermore, derived subcomponents of cardiac or respiratory cycles are accrued over multiple cycles and comparison analyzed to identify changes reflective of deviations or trends or both within the subcomponent indicating physiological trends or deviations.

The invention further comprises comparing the subcomponents of the subject with corresponding subcomponents of other individuals for the purpose of determining physiological differences.

PCT/US2007/020473 filed Sep. 21, 2007, entitled “Apparatus and Method for Non-invasive Thoracic Radio Interrogation”; U.S. Patent application Nos. 60/846,403 entitled “Method and Apparatus for Non-Invasive Bio Impedance Determination”, filed Sep. 21, 2006; U.S. Provisional application No. 60/846,402 entitled “Method for Conditioning Radio Signal Returns from Thoracic Components for Extractions of Cardiopulmonary Data”, filed Sep. 21, 2006; U.S. Provisional application No. 60/973,985, entitled “Apparatus and Method for Non-Invasive Thoracic Radio Interrogation”, filed Sep. 20, 2007; PCT/US2007/020492 filed Sep. 21, 2007, entitled “Antenna for Thoracic Radio Interrogation”; U.S. Provisional application No. 60/846,408 entitled “Transducer-antenna-probe for Thoracic Radio Interrogation”, filed Sep. 21, 2006, and U.S. Provisional Application No. 60/910,394, entitled “Antenna for Thoracic Radio Interrogation”, filed Apr. 5, 2007, and U.S. Provisional Application No. 60/973,970, entitled “Antenna for Thoracic Radio Interrogation”, filed Sep. 20, 2007, are also all incorporated by reference herein in their entireties.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A method of evaluating medical state of a subject comprising the steps of:
providing a radio frequency interrogation interference signal from a subject, the radio frequency interrogation interference signal being low frequency components of reflections of a radio interrogation signal transmitted into the thorax of the subject; and
determining at least one cardiac or respiratory characteristic of the subject from radio frequency interrogation interference signal.

2. The method of claim 1 wherein the determining step comprises the steps of:
identifying at least one cardiac or respiratory cycle of the subject in the radio frequency interrogation interference signal; and
identifying the at least one characteristic in the at least one cycle.

3. The method of claim 2 further comprising the steps of:
identifying the at least one characteristic from a subsequent cycle: and
comparing the characteristics to determine changes of the characteristic over time.

4. The method of claim 2 further comprising the steps of:
identifying the at least one characteristic from a subsequent cycle: and
averaging the characteristics.

5. The method of claim 2 wherein the determining step comprises the steps of:
dividing the radio frequency interrogation interference signal into time segments containing several cardiac cycles; and
identifying at least one respiratory event from changes amplitude of the radio frequency interrogation interference signal over the segment.

6. The method of claim 2 wherein the determining step comprises the steps of
dividing the radio frequency interrogation interference signal into time segments containing several cardiac cycles; and
determining the at least one respiratory characteristic changes within each cardiac cycle.

7. The method of claim 1 wherein the determining step comprises the steps of:
identifying the at least one characteristic from changes in slope of the radio frequency interrogation interference signal over the at least one cycle.

8. The method of claim 1 wherein the determining step comprises the steps of:
dividing the radio frequency interrogation interference signal into time segments containing several cardiac cycles; and
extracting a respiratory cycle component from each of the cardiac cycles of a first segment to provide a resultant cardiac component of the radio frequency interrogation interference signal over the first segment; and
determining at least one cardiac characteristic from the resultant cardiac component of the first segment of the radio frequency interrogation interference signal.

9. The method of claim 7 further comprising the step of deriving at least one cardiac or respiratory characteristic from within each cardiac cycle of the resultant cardiac component of the first segment.

10. The method of claim 8 further comprising the steps of:
deriving the at least one characteristic from another cardiac component of a second segment of the radio frequency interrogation interference signal; and
comparing the derived characteristics from the first and second segments to identify changes.

11. The method of claim 8 further comprising the steps of:
dividing the radio frequency interrogation interference signal into time segments, each time segment containing at least one respiratory cycle; and
extracting a respiratory cycle component from a first segment of the radio frequency interrogation interference signal over the first segment; and
determining at least one cardiac or respiratory characteristic from the resultant respiratory component of the first segment of the radio frequency interrogation interference signal.

12. The method of claim 11 further comprising the steps of:
deriving the at least one characteristic from another respiratory component of a second segment of the radio frequency interrogation interference signal; and
comparing the derived characteristics from the first and second segments to identify changes.

13. The method of claim 1 wherein the determining step comprising calculating at least respiration rate from the radio frequency interrogation interference signal.

14. The method of claim 1 wherein the determining step comprises calculating heart rate of the subject from the radio frequency interrogation interference signal.

15. The method of claim 14, wherein the determining step further comprises the step of calculating a value representative of stroke volume of the subject from the radio frequency interrogation interference signal.

16. The method of claim 15, wherein the determining step further comprises the step of calculating a value representative of cardiac output of the subject from the heart rate and stroke volume.

17. The method of claim 15, further comprises the step of calculating a ratio of change in amplitude with respect to change in time between opposite, sequential extrema in one cardiac cycle of the radio frequency interrogation interference signal.

18. The method of claim 17, further comprising the steps of:
repeating the step of calculating the ratio with respect to several sequential cardiac cycles of the radio frequency interrogation interference; and
averaging the ratios.

19. The method of claim 1, further comprising the step of identifying at least one cardiac or respiratory characteristic within one cardiac cycle of the radio frequency interrogation interference signal.

20. The method of claim 1 further comprising the step of monitoring the characteristic for change over time to identify an overall condition or changes in overall condition or both of the subject.

21. The method of claim 20 further comprising the step of at least periodically outputting a signal related to overall condition to the subject.

22. The method of claim 20 further comprising the step of outputting an alarm signal when overall condition of the subject falls below a predetermined value.

23. The method of claim 20 further comprising the step of quantifying the overall condition of the subject based on the characteristic.

24. The method of claim 23 wherein the quantifying step comprises quantifying cardiac output, stroke volume, respiratory rate, heart rate, a cardiac wave event or other cardiac or respiratory characteristic or some combination of characteristics and further comprising the step of comparing the quantified values with prior values of the subject or with predetermined nominal values applicable widely to subjects.

25. A method of processing cardio-respiratory/pulmonary data obtained by transmission of a radio interrogation signal into a subject torso and capturing reflections of the radio interrogation signal from various tissue of the torso comprising the steps of:
extracting time varying components from the captured reflections of the radio interrogation signal as a radio interrogation impedance signal; and
extracting at least one cyclical cardio-respiratory component from the radio interrogation impedance signal.
